(19) United States
(12) Plant Patent Application Publication
(10) Pub. No.: US 2019/0269058 P1
(43) Pub. Date: Aug. 29, 2019

(54) Lavandula plant named 'FW RADIANCE'

(50) ;

(71) Applicant: PLANT GROWERS AUSTRALIA, Wonga Park (AU)

(72) Inventor: Steven EGGLETON, Wandin North (AU)

(73) Assignee: PLANT GROWERS AUSTRALIA, Wong Park (AU)

(21) Appl. No.: 16/501,145

(22) Filed: Feb. 25, 2019

(30) Foreign Application Priority Data
Feb. 26, 2018 (AU) 2018/039

(51) Int. Cl. A01H 6/50

(52) U.S. Cl. PLT/445

(57) ABSTRACT

A new and distinct cultivar of Lavandula plant named ‘FW Radiance’, characterized by its compact and broadly upright plant habit; freely branching growth habit, dense and bushy appearance; freely flowering habit; purple-colored flowers with medium to large red purple-colored sterile flower bracts arranged on short terminal spikes; and good garden performance.

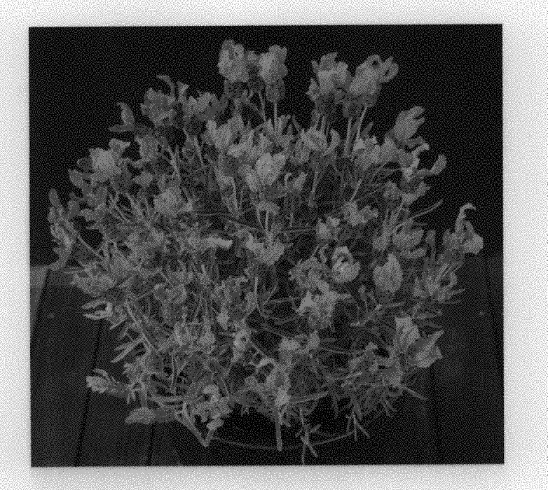

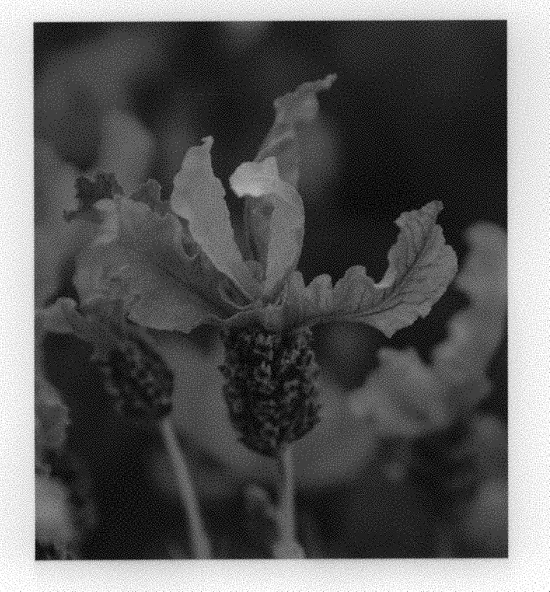

Botanical designation: Lavandula stoechas pedunculata.

Cultivar denomination: ‘FW RADIANCE’.

### CROSS-REFERENCED TO CLOSELY-RELATED APPLICATIONS

Applicant: Steven Eggleton

Title: Lavandula Plant Named ‘FW Spellbound’

Filed: Feb. 25, 2019, concurrently with this application

Applicant: Steven Eggleton

Title: Lavandula Plant Named ‘FW Whimsical’

Filed: Feb. 25, 2019, concurrently with this application

This application claims priority to the Australia Plant Breeder's Rights application number 2018/039, filed Feb. 26, 2018.

### BACKGROUND OF THE INVENTION

The present invention relates to a new and distinct Lavandula plant, botanically known as Lavandula stoechas pedunculata, commonly referred to as Spanish Lavender and hereinafter referred to by the name ‘FW Radiance’.

The new Lavandula plant is a product of a planned breeding program conducted by the Inventor in Wonga Park, Victoria, Australia. The objective of the breeding program was to develop new compact and freely-flowering Lavandula plants with good garden performance and attractive plant form and flower coloration.

The new Lavandula plant originated from a cross-pollination in October, 2009 of Lavandula stoechas pedunculata ‘Papillon’, not patented, as the female or seed, parent with Lavandula stoechas pedunculata ‘Strawberry Ruffles’, disclosed in U.S. Plant Pat. No. 22,490, as the male, or pollen, parent. The new Lavandula plant was discovered and selected by the Inventor as a single flowering plant within the progeny of the stated cross-pollination in a controlled greenhouse environment in Wonga Park, Victoria, Australia in October, 2014.

Asexual reproduction of the new Lavandula plant by softwood terminal cuttings in a controlled greenhouse environment in Wonga Park, Victoria, Australia since December, 2014 has shown that the unique features of this new Lavandula plant are stable and reproduced true to type in successive generations.

### SUMMARY OF THE INVENTION

Plants of the new Lavandula have not been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of the new Lavandula plant. These characteristics in combination distinguish ‘FW Radiance’ as a new and distinct Lavandula plant:

1. Compact and broadly upright plant habit.

2. Freely branching growth habit, dense and bushy appearance.

3. Freely flowering habit.

4. Purple-colored flowers with medium to large red purple-colored sterile flower bracts arranged on short terminal spikes.

5. Good garden performance.

Plants of the new Lavandula differ primarily from plants of the female parent, ‘Papillon’, in the following characteristics:

1. Plants of the new Lavandula are more upright than and not as spreading as plants of ‘Papillon’.

2. Plants of the new Lavandula have broader sterile flower bracts than plants of ‘Papillon’.

3. Plants of the new Lavandula and ‘Papillon’ differ in sterile flower bract color as flowers of plants of the new Lavandula have red purple-colored sterile flower bracts whereas flowers of plants of ‘Papillon’ have mauve-colored sterile flower bracts.

Plants of the new Lavandula differ primarily from plants of the male parent, ‘Strawberry Ruffles’, in the following characteristics:

1. Plants of the new Lavandula have shorter inflorescences than plants of ‘Strawberry Ruffles’.

2. Plants of the new Lavandula have narrower sterile flower bracts than plants of ‘Strawberry Ruffles’.

3. Plants of the new Lavandula and ‘Strawberry Ruffles’ differ in sterile flower bract color as flowers of plants of the new Lavandula have brighter red purple-colored sterile flower bracts than flowers of plants of ‘Strawberry Ruffles’.

4. Plants of the new Lavandula have longer peduncles than plants of ‘Strawberry Ruffles’.

Plants of the new Lavandula can be compared to plants of Lavandula stoechas pedunculata ‘FW Spellbound’, disclosed in a U.S. Plant Patent application filed concurrently. Plants of the new Lavandula and ‘FW Spellbound’ differ primarily in flower color as flowers of plants of the new Lavandula are purple in color with red purple-colored sterile flower bracts whereas flowers of plants of ‘FW Spellbound’ are dark violet in color with purple violet-colored sterile flower bracts.

Plants of the new Lavandula can be compared to plants of Lavandula stoechas pedunculata ‘FW Whimsical’, disclosed in a U.S. Plant Patent application filed concurrently. Plants of the new Lavandula and ‘FW Whimsical’ differ primarily in flower color as flowers of plants of the new Lavandula are purple in color with red purple-colored sterile flower bracts whereas flowers of plants of ‘FW Whimsical’ are light purple in color with light red purple-colored sterile flower bracts.

Plants of the new Lavandula can be compared to plants of the Lavandula stoechas ‘IBPR910-2’, disclosed in U.S. Plant Pat. No. 25,525. In side-by-side comparisons, plants ofthe new Lavandula differ primarily from plants of ‘IBPR910-2’ in the following characteristics:

1. Plants of the new Lavandula have shorter inflorescences than plants of ‘IBPR910-2’.

2. Plants of the new Lavandula have narrower sterile flower bracts than plants of ‘IBPR910-2’.

3. Sterile flower bracts of plants of the new Lavandula are more outwardly spreading than and not as upright as sterile flower bracts of plants of ‘IBPR910-2’.

4. Margins of sterile flower bracts of plants of the new Lavandula are not as undulate as margins of sterile flower bracts of plants of ‘IBPR910-2’.

Plants of the new Lavandula can also be compared to plants of the Lavandula stoechas ‘Bentley’, disclosed in U.S. Plant Pat. No. 22,709. In side-by-side comparisons, plants of the new Lavandula differ primarily from plants of ‘Bentley’ in the following characteristics:

1. Plants of the new Lavandula have longer sterile flower bracts than plants of ‘Bentley’.

2. Plants of the new Lavandula and ‘Bentley’ differ in sterile flower bract color as flowers of plants of the new Lavandula have darker red purple-colored sterile flower bracts than flowers of plants of ‘Bentley’.

3. Plants of the new Lavandula have longer peduncles than plants of ‘Bentley’.

### BRIEF DESCRIPTION OF THE PHOTOGRAPHS

The accompanying photographs illustrate the overall appearance of the new Lavandula plant showing the colors as true as it is reasonably possible to obtain in colored reproductions of this type. Colors in the photographs may differ slightly from the color values cited in the detailed botanical description which accurately describe the colors of the new Lavandula plant.

The photograph on the first sheet is a side perspective view of a typical flowering plant of ‘FW Radiance’ grown in a container.

The photograph on the second sheet is a close-up view of typical inflorescences of ‘FW Radiance’.

### DETAILED BOTANICAL DESCRIPTION

Plants used in the aforementioned photographs and following detailed description were grown in 20-cm containers during the spring in an outdoor nursery in Wonga Park, Victoria, Australia and under cultural practices typical of commercial Lavandula production. Plants were pinched two times and were one year old when the photographs and description were taken. During the production of the plants, day temperatures ranged from 10° C. to 40° C. and night temperatures ranged from −1° C. to 20° C. In the following description, color references are made to The Royal Horticultural Society Colour Chart, 2015 Edition, except where general terms of ordinary dictionary significance are used.
Botanical classification: Lavandula stoechas pedunculata ‘FW Radiance’.
Parentage:
   Female, or seed, parent.—Lavandula stoechas pedunculata ‘Papillon’, not patented.
   Male, or pollen, parent.—Lavandula stoechas pedunculata ‘Strawberry Ruffles’, disclosed in U.S. Plant Pat. No. 22,490.
Propagation:
   Type.—Terminal softwood vegetative cuttings.
   Time to initiate roots, summer.—About ten days at temperatures about 24° C.
   Time to initiate roots, winter.—About twelve days at temperatures about 21° C.
   Time to produce a rooted young plant, summer.—About 24 to 28 days at temperatures ranging from 23° C. to 26° C.
   Time to produce a rooted young plant, winter.—About 35 to 40 days at temperatures ranging from 15° C. to 18° C.
   Root description.—Fine, fibrous; typically white to light brown in color, actual color of the roots is dependent on substrate composition, water quality, fertilizer type and formulation.
   Rooting habit.—Freely branching; medium density.
Plant description:
   Plant and growth habit.—Herbaceous perennial; compact and broadly upright plant habit; roughly globular to broadly ovate in overall shape; moderately vigorous growth habit; moderate growth rate; flowers arranged in verticillasters on terminal spikes.
   Plant height, soil level to top of foliar plane.—About 16.3 cm.
   Plant height, soil level to top of floral plane.—About 22.6 cm.
   Plant width.—About 21.3 cm.
   Lateral branch description.—Quantity per plant: About nine primary lateral branches each with about ten secondary branches developing per plant during the flowering season. Length: About 6.5 cm. Diameter: About 1.5 mm. Internode length: About 2.9 cm. Strength: Moderately strong. Aspect: Upright to somewhat outwardly. Texture and luster: Densely tomentose; slightly glossy. Color, developing: Close to 144B to 144C; at the internodes, between 143C and 144B. Color, developed: Close to 199B and 199C.
   Leaf description.—Arrangement: Opposite, simple; sessile. Length: About 3.2 cm. Width: About 5 mm. Shape: Narrowly oblanceolate. Apex: Acute. Base: Cuneate. Margin: Entire; slightly to moderately revolute. Texture and luster, upper surface: Densely pubescent; matte. Texture and luster, lower surface: Moderately to densely pubescent; matte. Fragrance: Strongly aromatic, pungent. Venation pattern: Pinnate. Color: Developing leaves, upper surface: Close to 137B. Developing leaves, lower surface: Close to 138B. Fully expanded leaves, upper surface: Close to NN137B to NN137C; venation, close to 138A. Fully expanded leaves, lower surface: Close to 137B; venation, close to 138B.
Flower description:
   Flower type, arrangement and habit.—Small single salverform flowers arranged in verticillasters on terminal spikes; freely flowering habit with about 115 flowers developing per inflorescence and about 3,850 flowers developing per plant; flowers with two-lobed upper lip and three-lobed lower lip; flowers face mostly outwardly on the spike.
   Natural flowering season.—Relatively long flowering period; continuous from late spring into the summer in The Netherlands.
   Flower longevity on the plant.—Individual flowers last about 50 days on the plant; flowers not persistent.
   Fragrance.—None detected.
   Flower buds.—Length: About 7 mm. Diameter: About 2 mm. Shape: Elliptic. Texture and luster: Moderately tomentose; matte. Color: Close to 143B; distally, tinged with close to N186C; at the apex, close to N186A.
   Inflorescence height.—About 4.9 cm.
   Inflorescence diameter.—About 1.8 cm.
   Flower diameter.—About 7 mm.
   Flower depth (height).—About 1.1 cm.
   Petals.—Quantity and arrangement: Upper lip, two-lobed and lower lip, three-lobed. Length, upper lip: About 1 cm. Length, lower lip: About 9 mm. Width, upper lip: About 2 mm. Width, lower lip: About 2 mm. Shape: Roughly spatulate; lower 70% fused into a tube. Apex: Obtuse, rounded. Margin: Entire. Texture and luster, upper (inner) surface: Smooth, glabrous; slightly velvety; matte. Texture and luster, lower (outer) surface: Smooth, glabrous; slightly velvety; slightly glossy. Color: When opening, upper (inner) and lower (outer) surfaces: Close to N186A. Fully opened, upper (inner) surface: Close to between N92A and N186B; throat, close to 86A to 86B; color becoming closer to N186A with development. Fully opened, lower (outer) surface: Close to between N92A and N186B; tube, close to 86C and proximally, close to N87D; color becoming closer to N186A with development.
   Basal flower bracts.—Quantity and arrangement: Each group of seven flowers is subtended by a single basal flower bract. Length: About 1 cm. Width: About 9 mm. Shape: Broadly rhomboidal. Apex: Broadly aristate. Base: Cuneate. Margin: Entire. Texture and luster, upper and lower surfaces: Sparsely tomentose; matte. Color, upper surface: Close to 187C; venation, close to 146B. Color, lower surface: Close to 187C; venation, close to between N186B and N200A.
   Sterile flower bractsQuantity and arrangement: Four sterile flower bracts at apex of spike. Length: About 2.2 cm. Width: About 9 mm. Shape: Narrowly elliptic to narrowly oblong to narrowly obovate. Apex: Acute. Base: Cuneate. Margin: Entire; undulate. Texture and luster, upper and lower surfaces: Sparsely tomentose; matte. Color, upper surface: Close to N82B; main vein, tinged with close to N186B. Color, lower surface: Close to N82B; main vein, close to 187A.
   Sepals.—Quantity and arrangement: Five, fused into a campanulate tube. Calyx length: About 7 mm. Calyx diameter: About 2 mm. Shape: Lanceolate. Apex: Acute. Margin: Entire. Texture and luster, upper (inner) and lower (outer) surfaces: Densely pubescent; moderately glossy to glossy. Color: When opening, upper (inner) surface: Close to 145A; venation, close to 143A. When opening, lower (outer) surface: Close to 143B; distally, tinged with close to N186C. Fully opened, upper (inner) surface: Close to 145A; venation, close to 143A. Fully opened, lower (outer) surface: Close to 143A.
   Peduncles.—Length: About 5.3 cm. Diameter: About 2 mm. Aspect: Mostly upright. Strength: Strong. Texture and luster: Densely tomentose; matte. Color: Close to 144A; distally, tinged with close to 176A.
   Reproductive organs.—Stamens: Quantity per flower: Four. Filament length: About 1 mm. Filament color: Close to 84D. Anther shape: Reniform. Anther length: About 0.75 mm. Anther color: Close to 21B to 21C. Pollen amount: Scarce to moderate. Pollen color: Close to 14A to 14B. Pistils: Quantity per flower: One. Pistil length: About 4 mm. Stigma diameter: About 0.5 mm. Stigma shape: Club-shaped. Stigma color: Close to N186A. Style length: About 3.5 mm. Style color: Close to 85C. Ovary color: Close to 143A to 143B.
   Seeds and fruits.—To date, seed and fruit production has not been observed on plants of the new Lavandula.
Pathogen & pest resistance: To date, plants of the new Lavandula have not been noted to be resistant to pathogens and pests common to Lavandula plants.
Garden performance: Plants of the new Lavandula have exhibited good garden performance and to tolerate rain and wind and temperatures ranging from −2° C. to 42° C.

It is claimed:

1. A new and distinct Lavandula plant named ‘FW Radiance’ as illustrated and described.
